(19) United States
(12) Patent Application Publication
(10) Pub. No.: US 2004/0199256 A1
(43) Pub. Date: Oct. 7, 2004

(54) Support device for supporting between spinal vertebrae

(76) Inventor: Chao-Jan Wang, Hsinchu (TW)

Correspondence Address: Alan D. Kamrath, Rider Bennett, LLP, Suite 2000, 333 South Seventh Street, Minneapolis, MN, 55402, US

(21) Appl. No.: 10/407,576

(22) Filed: Apr. 4, 2003

(51) Int. Cl. A61F 2/44; A61F 2/28

(52) U.S. Cl. 623/17.12; 623/23.51; 428/320.2

(57) ABSTRACT

A supporting device for osteopathy includes a liquid material and a support frame which includes a plurality of support boards or support links connected with each other. The liquid material is attached onto the support frame and solidified so as to form a support frame that has support boards or support links are crossed over each other.

### FIELD OF THE INVENTION

The present invention relates to a support device having a frame for supporting between spinal vertebrae.

### BACKGROUND OF THE INVENTION

Conventional material used in osteopathy for filling damaged area of bones is in a form a powder, particles or bone cement such as poly methyl methacrylate or bone substitute such as calcium sulfate or calcium phosphate. Nevertheless, there are experienced that these conventional types of material often generate heat or debris which could press on the nerves located close to the damaged areas of the bones.

In particularly, in supporting vertebrae in cervical vertebrae, lumbar vertebrae of the spinal, the existed method used in osteopathy is to insert a supporting device between the spinal vertebrae. Material is filled in chambers in the supporting device which is then inserted between the vertebrae. The supporting device is made of metal which is heavy in weight and could sink between the vertebrae. The material is mixed before being moved into the patient's body so that the heat is generated due to the mixture could affect the tissues of the body. If a piece of artificial bone is inserted between the spinal vertebrae, because the artificial bone is fragile in nature so that it is a problem to be carefully monitored if the artificial bone is broken.

The present invention intends to provide a supporting device used in osteopathy and the device can be composed of liquid material and a frame. The liquid material can be connected to the frame and includes a cruciform device for supporting between spinal vertebrae.

### SUMMARY OF THE INVENTION

In accordance with one aspect of the present invention, there is provided a supporting device that comprises a support frame comprising a plurality of support boards or support links which are connected perpendicularly with each other and the support boards or links are connected between a front board and a rear board. A liquid material is coated on the support frame and solidified.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 shows a preferred embodiment of the supporting device of the present invention;

FIGS. 2a and 2b show the liquid material and the support frame;

FIG. 3 shows a side view of the support frame;

FIGS. 4a and 4b show the second and the third preferred embodiment of the present invention;

FIGS. 4c and 4d show embodiments of the mediate portion of the present invention, and

FIGS. 5a and 5b show the supporting device is inserted between the spinal vertebrae.

### DETAILED DESCRIPTION OF THE PREFERRED EMBODIMENT

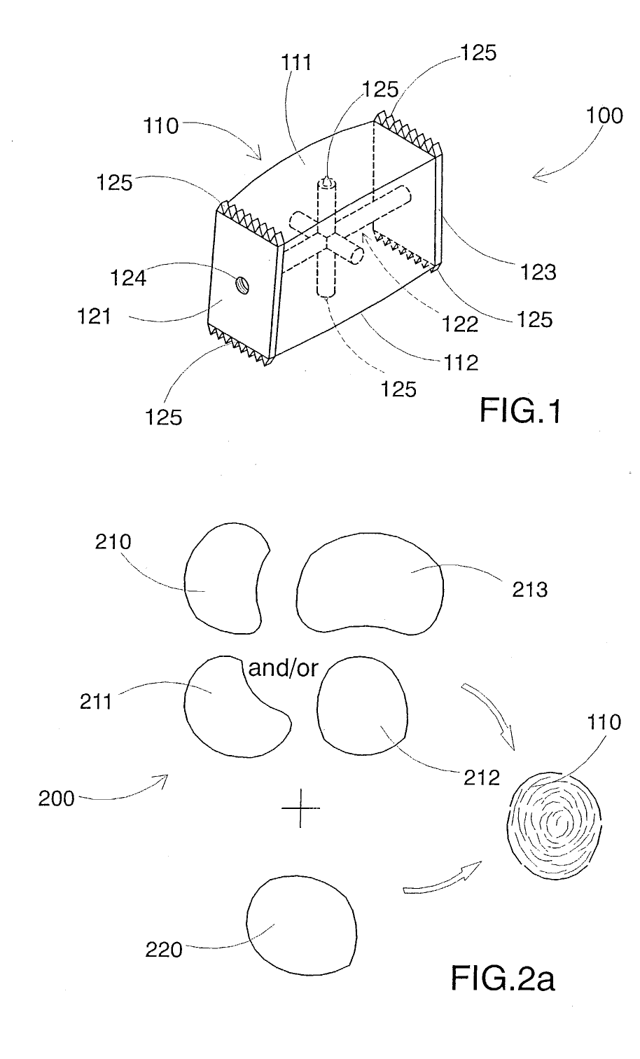

Referring to FIG. 1, the supporting device 100 of the present invention is a final form 110 which is formed by a liquid material which is solidified to becomes the final form 110. The reference 120 is a support frame of the first embodiment of the supporting device 100 and reference 111 represents a top surface of the supporting device 100 and which contacts the a spinal vertebra. Reference 112 is a bottom surface which is located in opposite to the top surface. Reference 121 is a front board of the support frame 120, 122 is a mediate portion of the support frame 120, and 123 is a rear board of the support frame 120. A threaded hole 124 is defined in the front board 121 and a threaded hole can be drilled in the rear board 123 and which is not shown in the drawings. A plurality of teeth 125 are defined in top and bottom edge of the front board 121, mediate portion 122 and the rear board 123, the teeth 125 are to be contact with spinal vertebrae. The supporting device 100 is an artificial member inserted between the spinal vertebrae so as to re-locate the spinal vertebrae in correct positions.

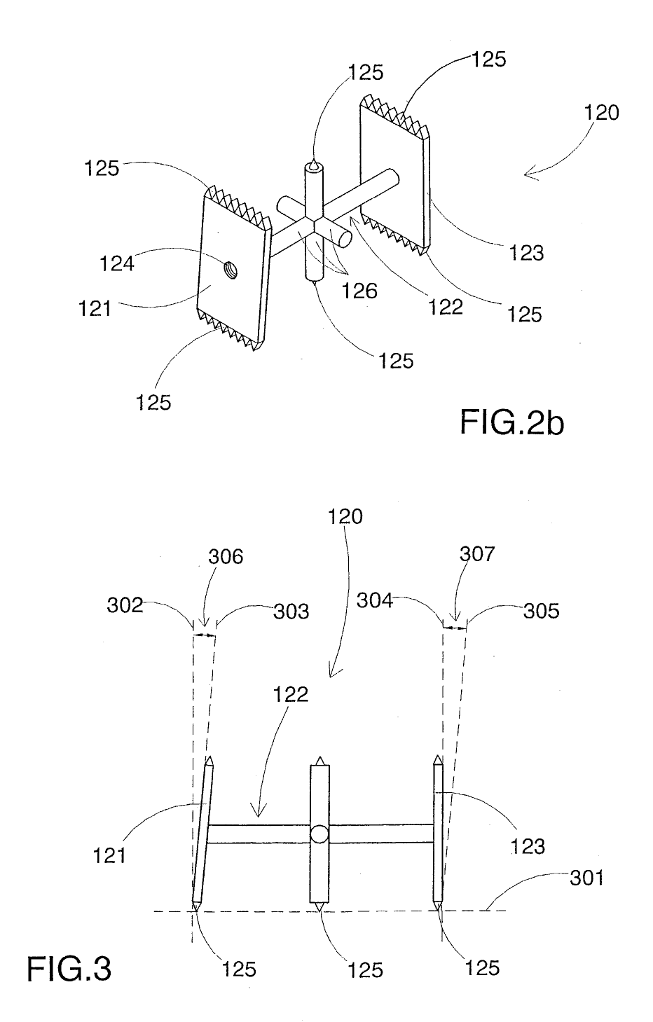

In FIG. 2a, the liquid material 110 of the supporting device 100 is made by one or by mixing several materials 200 such as hydroxy apatite, calcium phosphate compounds 211, calcium sulfate compounds 212 or human bones 213 with medicine or liquid 220 such as water, oil or gel at proper rates. The mixture can be put for a period of time, adding pressure or heat, or filled in a mold to form a specific shape.

In FIG. 2b, the support frame 120 includes a front board 121, a mediate portion 122 and a rear board 123, wherein the front board 121 has a threaded hole 124 for a tool (not shown) to be connected therewith. The mediate portion 122 includes three support links 126 which are connected perpendicularly with each other so as to form a cruciform structure. The front board 121 and the rear board 123 are connected by the mediate portion 122. Teeth 125 are defined in those portions to be in contact with spinal vertebrae of the front board 121, the rear board 123 and the mediate portion 122. The teeth 125 assist the supporting device 100 to be fixed in correct position.

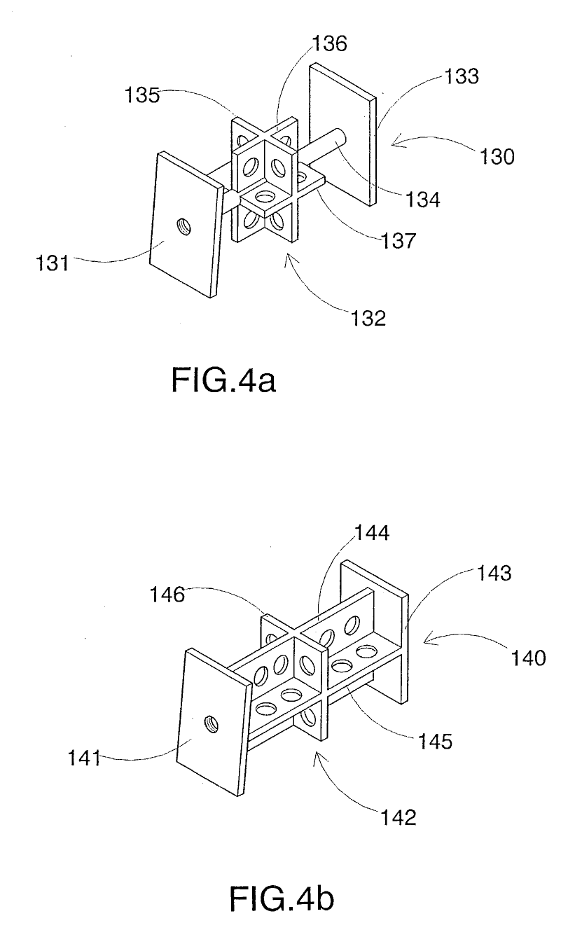

In FIG. 3, an imaginary line 301 is connected between the tips of the teeth 125 at the bottom edge of the frame 120, a tangent line 302 that passes the lower edge the front board 121 and perpendicular to the imaginary line 301, a line 303 that passes the outer surface of the front board 121, a line 304 that passes the outer surface of the rear board 123, and a line 305 that passes a lower edge of the rear board 123 and being parallel with the line 303. If the front board 121 is connected to the mediate portion 122 at its central point, the front board 121 is inclined 3 to 5 degrees 306 toward the right as shown by the angle between the lines 302 and 303, or is perpendicular to the mediate portion 122. If the rear board 123 is connected to the mediate portion 122 at its central point, the rear board 123 is inclined 3 to 5 degrees 307 toward the left as shown by the angle between the lines 304 and 305, or is perpendicular to the mediate portion 122. The inclined angles 306 and 307 are the same as the lordosis of the spine, or when the supporting device is inserted in the cervical vertebrae, the front board 121 is in contact with the vertebrae body end-plate of the adjacent vertebrae.

As shown in FIG. 1, the top surface 111 and the bottom surface 112 can be a convex surface which matches with the curvature of the bottom surface of the spinal vertebrae, or the top surface 111 and the bottom surface 112 can be a flat surface to provide a full contact area.

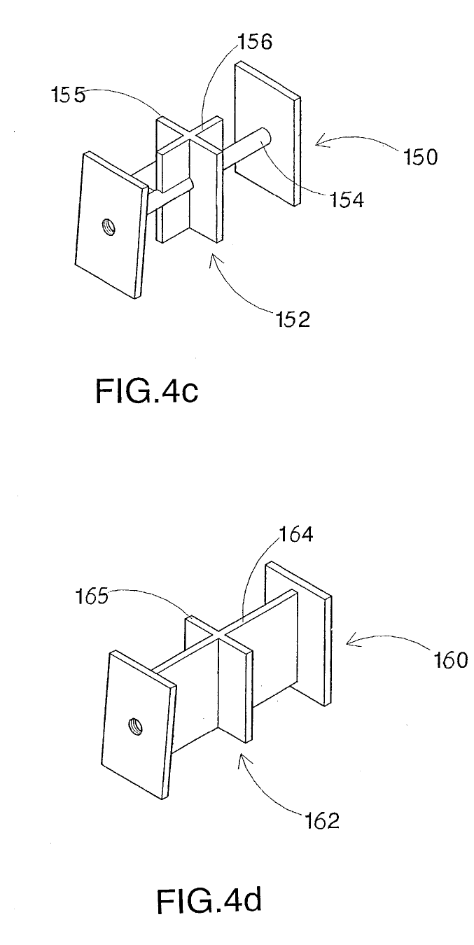

In FIG. 4a, in the second embodiment 130, the mediate portion 132 is a support link 134 which is connected between the front board 131 and the rear board 133. Three support boards 135, 136 and 137 are connected between the front board 131 and the rear board 133, wherein the support board 135 is parallel with the front board 131 and the rear board 133, the support board 137 is perpendicular to the support boards 135 and 136. The edges that are in contact with the vertebrae may have teeth or holes defined therein.

In FIG. 4b, in the third embodiment 140, the mediate portion 142 is composed of three support boards 144, 145 and 146 which are connected perpendicularly with each other and are connected between the front board 141 and the rear board 143. The support board 145 is parallel with the front board 141 and the rear board 143. The edges that are in contact with the vertebrae may have teeth or holes defined therein.

The mediate portions 152, 162 as shown in FIGS. 4c and 4d are similar to those shown in FIGS. 4a and 4b. The support boards or the support links may have holes so that the human bone can be firmly attached to the support boards when it grows. The support boards 157, 166 can also be omitted so that the cells of the bones will not grow in vertical directions.

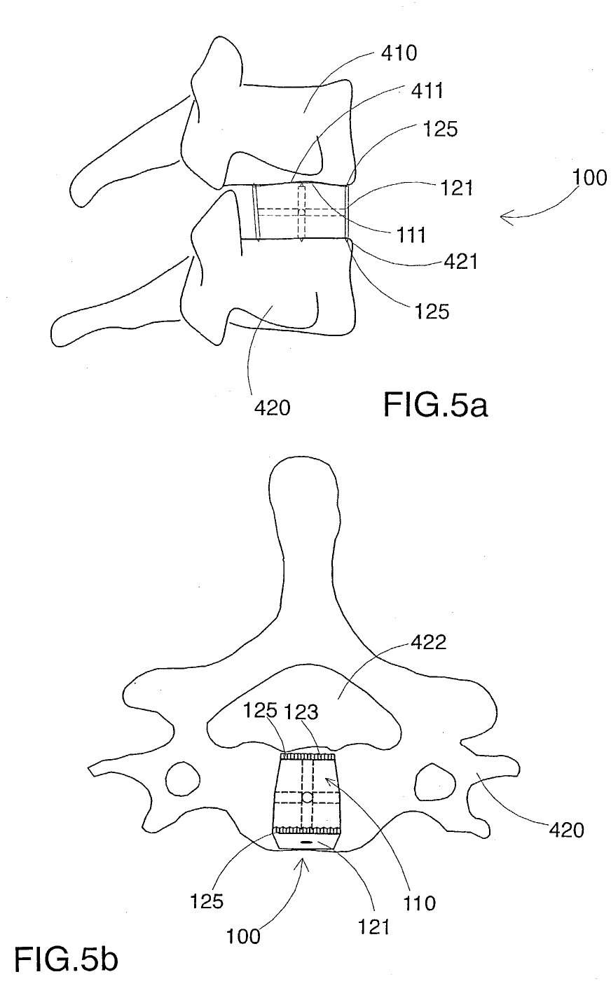

FIGS. 5a and 5b show that the supporting device 100 can be inserted between a top cervical vertebra 410 and a low cervical vertebra 420. The top cervical vertebra 410 has a bottom surface 411 which has a concave curvature and the top surface 111 of the supporting device 100 is shaped to match with the concave curvature in the bottom surface 411 of the low vertebra 410 so as to obtain a maximum support area and force.

The low vertebra 420 includes an end-plate 421 and there is a cervical curvature defined in the cervical vertebra so that the low vertebra 420 has a tilt angle of 3 to 5 degrees relative to the top vertebra 410. The angle for the front board 121 of the supporting device 100 is matched with the tilt angle of the top vertebra 410 and the low vertebra 420. The teeth 125 may contact and penetrate the front edge of the end plates of the top vertebra 410 and the low vertebra 420. This makes the supporting device 100 to be securely positioned and will not sink into the low vertebra 420.

In FIG. 5b, the support board 123 of the supporting device 100 allows the spinal nerves to pass through the vertebral foramen 422. The front board 121 and the rear board 123 securely clamp the material 110 so that it is not likely to be broken. The teeth 125 reinforce the positioning of the supporting device between the top vertebra 410 and the low vertebra 420, so as to prevent the supporting device from dropping.

While we have shown and described the embodiment in accordance with the present invention, it should be clear to those skilled in the art that further embodiments may be made without departing from the scope of the present invention.

What is claimed is:

1. A supporting device comprising:
a liquid material which can be solidified;
a support frame comprising a plurality of support boards or support links which are crossed over each other, the liquid material coated on the support frame so that after the liquid material is solidified, the supporting device has crossed support boards or support links.

2. The device as claimed in claim 1, wherein the support frame includes a front board, a mediate portion and a rear board, the solidified material being coated onto the mediate portion which is clamped between the front board and the rear board.

3. The device as claimed in claim 1, wherein the support frame has holes defined therethrough.

4. The device as claimed in claim 2, wherein the mediate portion includes a plurality of support boards and/or support links crossed over each other.

5. The device as claimed in claim 2, wherein the front board is connected to the mediate portion at a center point thereof and the front board is tilt at a tilt angle 3 to 5 degrees.

6. The device as claimed in claim 2, wherein the rear board is connected to the mediate portion at a center point thereof and the rear board is tilt at a tilt angle 3 to 5 degrees.

7. The device as claimed in claim 2, wherein the front board, the mediate portion and the rear board have teeth defined in a top edge and a bottom edge thereof.

8. The device as claimed in claim 2, wherein one of the front board and the rear board has a threaded hole defined therein.

9. The device as claimed in claim 1, wherein the liquid material of the supporting device is made by mixing human bone, hydroxy apatite, calcium phosphate compounds, or calcium sulfate compounds with liquid or gel.

10. The device as claimed in claim 1, wherein the solidified liquid material coated onto the support frame can be cubic items, cubic items with curve surfaces or trapezoid cubic items.
